(19) United States
(12) Patent Application Publication
(10) Pub. No.: US 2010/0286583 A1
(43) Pub. Date: Nov. 11, 2010

(54) Non-Woven Fabric

(75) Inventors: Manel Torres, London (GB); Paul Luckham, Middlesex (GB); Michael Musoke, London (GB)

Correspondence Address: WHITHAM, CURTIS & CHRISTOFFERSON & COOK, P.C., 11491 SUNSET HILLS ROAD, SUITE 340, RESTON, VA, 20190, US

(73) Assignee: Manel Torres, London (GB)

(21) Appl. No.: 11/993,762

(22) PCT Filed: Jun. 28, 2006
(86) PCT No.: PCT/GB2006/002382

§ 371 Date: Dec. 1, 2008

(30) Foreign Application Priority Data
Jun. 29, 2005 (GB) 0513350.9

(51) Int. Cl. A61F 13/00; C08L 53/02; C11D 3/382; A61K 47/32; A61L 9/04; D06P 5/00; C11D 3/37; B05D 1/10; D04H 1/00; B65D 85/00; B65D 25/40

(52) U.S. Cl. 602/45; 524/34; 524/505; 510/462; 514/772.4; 512/4; 8/467; 510/475; 427/427.4; 442/327; 206/525; 222/566

(57) ABSTRACT

This invention provides a composition for forming a fabric by spraying onto a supporting surface, the composition comprising fibers, a binder dissolved in a first carrier and a second carrier wherein the boiling point of the second carrier is at least 400 C and is higher than the boiling point of the first carrier. Further aspects of the invention include the fabric formed by spraying the composition and an article comprising the fabric, an apparatus and a method for spraying the composition.

### FIELD OF THE INVENTION

This invention relates to non-woven fabrics having a liquid incorporated therein, and in particular to a method of making such fabrics by spraying fibres onto a support member, and to a mixture of fibres, binder, first carrier and second carrier for use in the method of the invention.

### BACKGROUND

Fabrics are required in a wide variety of applications and industries, including for instance the cleaning and cosmetic industries where fabrics are used as applicators for various active agents. Fabrics are also useful in a number of medical applications. Most commonly fabrics are woven fabrics, although it is also known to produce non-woven fabrics. The industries which require fabrics have a need for the provision of fabrics by methods which are flexible and convenient, and in particular methods which allow fabrics to be formed quickly and easily, from a variety of different fabric materials and in a variety of different shapes.

Our publication WO 03/104540 discloses a composition comprising fibres, a binder and a diluent which can be sprayed onto a supporting surface to produce a non-woven fabric.

The Examples in WO 03/104540 describe a single phase composition prepared with ethyl acetate, acetone or methanol as a diluent.

Other diluents mentioned in the description are water, alcohols, ketones and esters having between 1 and 12 carbon atoms.

Substantially all of the diluent evaporates before the composition reaches the supporting surface and so the fabric formed comprises the fibres and binder with any optional additives but without any diluent.

Thus, this publication allows provision of non-woven fabrics containing a wide variety of fabric materials, by a simple and straightforward method. It also allows application of fabric to a wide variety of different surfaces and with almost unlimited scope for the contours of the fabric.

In recent years a range of products colloquially called “wet wipes” have become increasing popular for various cleaning applications. Wet wipes consist of a fabric impregnated with a liquid agent. They are convenient to use as they combine a cloth or applicator with an active agent saving time and effort for the consumer. Wet wipes must be stored in an airtight environment to prevent the liquid agent from evaporating.

Products that comprise a fabric with a liquid incorporated in it are also known for medical dressings and in the cosmetic field. For example, products combining a fabric with liquid nail varnish remover, cleanser or make-up remover are known.

It would be desirable to provide a convenient and flexible way of obtaining a fabric having a liquid incorporated therein.

### SUMMARY OF THE INVENTION

A first aspect of this invention provides a composition for forming a fabric by spraying onto a supporting surface, the composition comprising fibres, a binder, a first carrier in which the binder is dissolved and a second carrier wherein the boiling point of the second carrier is at least 40° C. and is higher than the boiling point of the first carrier.

A second aspect of this invention is an apparatus comprising a container containing the composition of the present invention which is capable of producing a spray of the composition from the container.

A third aspect of this invention is a non-woven fabric produced by spraying a composition of the present invention, wherein the fabric comprises the binder in solid form, fibres bound by the binder and second carrier in liquid form.

A fourth aspect of this invention is an article comprising a non-woven fabric according to the third aspect of this invention.

A fifth aspect of this invention is a method for producing a non-woven fabric comprising

a) providing a container capable of producing a spray containing a composition according to the present invention, and

b) spraying the composition onto a supporting surface thereby forming a non-woven fabric on the supporting surface.

The compositions of the present invention comprise a first carrier and a second carrier as the diluent for the binder and fibres. The first carrier has a lower boiling point than the second carrier and substantially all evaporates during spraying before the fabric is formed. However, not all of the second carrier evaporates and it becomes incorporated into the fabric. Thus the fabric is a non-woven fabric made up from solid binder and fibres having a liquid second carrier in the fabric.

Compositions of the present invention are very flexible and a wide range of additives can be included to make the products suitable for various different applications. For example, cleaning or cosmetic active agents can be included in the composition so that they are incorporated in the fabric produced to make the product suitable for cleaning or cosmetic applications such as hard surface cleaning or make-up removal.

The present invention is also useful in medical applications where moist dressings are often needed. An example of such a medical application is the emergency treatment of burns. Compositions of the present invention could be sprayed onto the affected area of skin to provide a wet cooling dressing. Pharmaceutical additives such as drugs can be incorporated into the composition to make the dressing more effective.

In a medical setting, the fabric could also be used to clean hard surfaces and could, for example, include a disinfectant that would make it suitable for killing bacteria, such as the MRSA bacteria. It could also be used to clean patients, for example, to disinfect an area of skin before an injection is given.

The container from which the composition is sprayed is generally sealed and can be sterilised during manufacture which means that the fabric produced would be sterile, other than the contact it has had with the atmosphere. This make is highly suitable for use in medical or cosmetic application.

Spraying is a very flexible way of forming a fabric as it allows a fabric of any size or shape to be created. The composition of the present invention can be sprayed using a spray gun, artist's airbrush or from an aerosol can.

### DETAILED DESCRIPTION OF THE INVENTION

The fabric of the present invention formed by spraying the composition of the present invention offers many new possibilities for cleaning, cosmetic and medical applications and can be used in any field where a fabric having a liquid incorporated therein could be used.

The “fabric” provided by the present invention is a non-woven textile material which is often felt-like or fleece-like in texture.

The composition of the present invention is sprayed onto a solid “supporting surface”. This can for instance be an item on which the fabric is to be used (such as a dirty surface) or a mould or other surface from which the fabric, once formed, can be easily separated or peeled away.

The invention can, for example, be used to provide a convenient oven cleaner. A fabric that has cleaning actives included in it can be produced by spraying onto the oven wall. The fabric can then be peeled off and discarded or used to wipe the area if more cleaning is required.

The composition of the present invention comprises a first carrier and a second carrier wherein the first carrier is more volatile than the second carrier. The second carrier becomes incorporated in the fabric as a liquid so must be a liquid at temperatures up to 40° C. The second carrier has a higher boiling point than the first carrier. Preferably the boiling point of the second carrier is at least 30° C. higher than the boiling point of the first carrier, more preferably at least 50 C higher, most preferably at least 60° C. higher.

The relative volatilities of the first carrier and the second carrier reflect the purpose of each of these components. The first carrier is a liquid in the composition before spraying. The first carrier, however, may have a boiling point below room temperature and to obtain liquid conditions, the composition can be stored at a high pressure. For example, the first carrier can be a propellant such as dimethyl ether. In such a case, the composition is stored in a pressurised container such as a metal cylinder colloquially known as an “aerosol can” under a pressure of above 1 and less than 20 atmospheres.

The purpose of the first carrier is to solubilise the binder in the composition so that the composition can be sprayed. If the binder were not soluble in the composition, there would be problems with forming a coherent fabric from the fibres and spraying of the composition. The first carrier then evaporates once the composition has been formed into a spray before the spray reaches the supporting surface and the fabric is formed. Once the first carrier has evaporated, the binder is left in solid form to bind together the fibres to form a fabric.

The first carrier is normally an organic solvent for example a C₂ to C₁₂ ether, such as diethyl ether, a C₁ to C₁₂ alcohol, a C₁ or C₃ to C₁₂ ketone a C₅ to C₁₂ hydrocarbon or a C₁ to C₁₂ ester. The first carrier may also be a propellant such as dimethyl ether or liquefied petroleum gas (which is a commercial mixture of propane and butane).

It is advantageous to have a first carrier which is a propellant as the first carrier will have a further function of propelling the spray. When the composition comprises a propellant, the composition should be stored in a pressurised container. When an opening is made in the container (usually at a nozzle for spraying) the propellant, with the rest of the composition, leaves the container in a form of a spray as a result of the energy stored in the pressurised propellant.

In contrast, the purpose of the second carrier is that it should not substantially evaporate during spraying and should become incorporated into the fabric. The second carrier is a liquid under ambient conditions (20° C. and 1 atmosphere) so giving the fabric a “wet” character.

The second carrier can be selected from the group consisting of water, ketones, alcohols, esters and ethers. Preferably, the second carrier is selected from the group consisting of water, acetone; ethanol, polyhydric alcohols, glycol ethers and mixtures thereof. Suitable polyhydric alcohols include ethylene glycol, propylene glycol, isoprene glycol, and dipropylene glycol, glycerol, 1,3-butylene glycol, sorbitol, diethylene glycol monoethyl ether and mixtures thereof.

One essential requirement of the composition is the relative boiling points of the first and second carriers so in any particular composition, the combination of carriers is important. Therefore, although acetone, for example, could potentially be a first carrier in one composition and a second carrier in another composition, in any single composition two different liquids are needed.

The second carrier should be selected according to the desired application of the fabric. For example, where the wet wipe is to be used as a facial cleansing wipe, the second carrier could comprise predominantly water. If the product produced is to be used to remove nail varnish, the second carrier could be predominantly acetone.

Preferably, the second carrier comprises water. In preferred embodiments, at least 30%, preferably at least 50% of the second carrier is water.

The first carrier and second carrier may or may not be miscible with each other. Where they are not miscible, the composition comprises an emulsion. An emulsifier can be included to facilitate formation of an emulsion.

The emulsifier is preferably designed to produce an emulsion of second carrier in first carrier. Surfactants are suitable emulsifiers, for example Span 85 or Tween 20. Mixtures of surfactant may also be used.

Having an emulsion of second carrier in first carrier allows the first carrier to evaporate easily on spraying leaving the second carrier to be incorporated into the fabric.

The first carrier usually comprises between 10 and 80% by weight of the composition based on the total weight preferably between 20 and 50%.

Usually the second carrier comprises between 5 and 60% by weight of the composition based on total weight, preferably between 10 and 50%.

Both the composition and the fabric of the present invention comprise a “binder” which is solid at ambient temperature and binds the sprayed material together, but is soluble in the first carrier and acts to bind the fibres into a fabric on spraying. Hence the binder and the first carrier are matched so that they are complementary. The binder should be substantially insoluble in the second carrier.

The requirement for binder to be soluble in the first carrier but not soluble in the second carrier applies under circumstances where both the first carrier and second carrier are liquid. For the first carrier, this may not be at normal atmospheric pressure (1 atmosphere), in particular where the first carrier is a propellant.

In the composition the binder is dissolved in the first carrier. Therefore, the binder needs to be soluble in the first carrier at the relative levels of these compounds present in the composition.

A binder should be selected so that it is soluble in the first carrier (which is preferably an organic volatile solvent) but not soluble in the second carrier (which is preferably a polar compound).

The binder may be polymeric and can be a homopolymer or a polymer of two or more monomers. The binder is preferably selected from the group consisting of, a thermoplastic elastomeric block copolymer such as styrene butadiene or styrene isoprene block copolymers or hydrogenated butadiene styrene or hydrogenated isoprene styrene block copolymers, polyvinyl acetate, polyvinyl butyrate, natural latex and polyvinyl alcohol or a mixture thereof. The binder must be a solid at the spraying temperature and to be soluble in the first carrier.

Both the composition and the fabric of the present invention comprise “fibres” of fabric material which are slender, elongated structures having an aspect ratio (ratio of length to diameter) of at least 3:1 and preferably at least 5:1, more preferably at least 10:1.

The fibres in the composition must be of a certain minimum length. Generally at least 80% of the fibres have a length of at least 0.02 mm. Preferably at least 90%, more preferably at least 95% and most preferably substantially all of the fibres in the composition have a length of at least 0.02 mm.

The fibres of the composition should not be too long since a composition comprising long fibres cannot be sprayed easily because the fibres block a nozzle. Generally, at least 80% (by weight) of the fibres have a length not more than 10 mm, preferably not more than 5 mm, more preferably not more than 1 mm, still more preferably not more than 0.5 mm, even more preferably not more than 0.25 mm, most preferably not more than 0.15 mm. Preferably, at least 90%, more preferably at least 95% and most preferably substantially all of the fibres have a length not more than 10 mm, preferably not more than 5 mm, more preferably not more than 1 mm, still more preferably not more than 0.5 mm, even more preferably not more than 0.25 mm, most preferably not more than 0.15 mm. In a preferred embodiment substantially all the fibres have a length in the range 0.02 to 0.15 mm.

The fibres should be dispersed in the composition. Where the first carrier and second carrier are not miscible and an emulsion is formed, the fibres may be dispersed in either or both phases. Where there is an emulsion, if hydrophilic fibres are used, the fibres will be preferentially dispersed throughout the second carrier whereas if hydrophobic fibres are used these will be dispersed preferentially throughout the first carrier. It has been found that particularly good results are obtained when a mixture of hydrophilic and hydrophobic fibres are used.

Both synthetic and natural fibres may be used in the composition. The fibres may be natural fibres, such as cotton, to give a fabric which is similar to cotton wool, or silk to give a smooth, cloth. Alternatively, synthetic fibres may be used, such as polypropylene, polyethylene or polyamide, which can give “fleece like” fabrics.

Generally the fibres in the composition are at least 10, 20, 30 or 40% (by weight of fibres) polymeric fibres. In a preferred embodiment most of the fibres in the composition are polymeric and organic, even though other fibre types may be used. Thus preferably at least 50, 60, 70 or 80%, especially at least 90%, of the fibres are polymeric. Most preferably only organic, polymeric fibres are used.

For the composition when sprayed to produce a non-woven fabric a sufficient binder to fibre ratio is usually desirable. For example the ratio of binder to fibres is preferably not greater than 5:1, more preferably not greater than 4:1 and even more preferably not greater than 3:1 and is preferably not less than 1:5, more preferably is not less than 1:4 and more preferably is not less than 1:3. Preferably, the ratio of fibres to binder is in the range 5:1 to 1:5, more preferably 3:1 to 1:3, even more preferably 2:1 to 1:2, most preferably 1.5:1 to 1:1.5.

The composition preferably comprises at least one additive. The additive may be selected so that it does not evaporate during spraying and can thus become incorporated into the fabric. The additive is usually a liquid active agent which is desirable in the product, for example, a cleaning agent, moisturiser, perfume, or drug. The additive may be soluble in the second carrier.

For different colours of spray-on fabric, dyed fibres may be used, or small quantities of dye can be added directly to the diluent. Food dyes are particularly suitable, but any dye soluble in the diluent can be used.

For example, if the spray-on fabric is required to adhere to a surface, an adhesive agent may also be incorporated. Adhesives used for spray contact photographic mounting are particularly suitable.

Other materials that could be added to the composition to be sprayed along with the fibres include:

Micro/nanocapsules containing:

Abrasive particles such as silica, titania, calcium carbonate etc

Oils: e.g. citronella, eucalyptus, neem etc

Perfumes

Drugs

Vitamins

Surfactants

Moisturisers

Natural antibiotics

Proteins

Health & beauty products such as: deodorants and antiperspirants

Preferably, at least 50%, 75%, 85% or 95% by weight of the total solids in the composition are fibres and binder.

The additives must be selected so that the fibres do not aggregate when in the composition. Some agents eg. dispersants such as surfactants may be added to prevent aggregation of fibres in the composition.

The choice of binder and other components also has an effect on the viscosity of the composition. If the viscosity is too high the composition will be difficult to spray. Preferably, the composition has a viscosity in the range 10 mPas to 100 Pas, more preferably in the range of 1 Pas to 10 Pas. The viscosity of the composition is measured using a concentric cylinder rheometer (Paar Physica Universal Dynamic Spectrometer [UDS] 200) at a temperature of 25° C.

It is not necessary to use only one type of fibre or first carrier or second carrier or binder in the composition and blends of more than one material for each component may be used. Additionally if the composition comprises additives one or more additives may be used in the same composition.

A second aspect of this invention is an apparatus comprising a container containing the composition of the present invention which is capable of producing a spray of the composition from the container.

The composition in the container may be at a pressure of above 1 and below 20 atmospheres and preferably between 3 and 20 atmospheres, more preferably between 5 or 8 and 15 atmospheres. The first carrier and second carrier should both be liquid under conditions that prevail in the container. Preferably, container has a nozzle with internal diameter of 0.05 to 2 mm, more preferably 0.1 to 1 mm.

A third aspect of this invention is a non-woven fabric produced by spraying a composition of the present invention, wherein the fabric comprises the binder in solid form, fibres bound by the binder and the second carrier in liquid form. The fabric preferably comprises at least 50% of the second carrier that was present in the composition before spraying and less than 10% of the first carrier that was present in the composition before spraying.

In fleece-like fabrics the fibres do not lie parallel to the surface.

Preferably, the fibres in the fabric are conjugated and are longer than the fibres in the composition. We believe that the fibres in the fabric are formed from shorter fibres in the composition joined by binder so that they partially overlap. The conjugated fibres are at least 1 mm, preferably 2 mm, more preferably 5 mm.

A fourth aspect of this invention is an article comprising a non-woven fabric according to the third aspect of this invention. Preferably the article is selected from a cleaning cloth, a cosmetic applicator or a medical dressing.

A fifth aspect of this invention is a method for producing a non-woven fabric comprising

a) providing a container capable of producing a spray a composition of the present invention, and

b) spraying the composition device onto a supporting surface thereby forming a non-woven fabric on the supporting surface.

During the spraying in step b) of the method, at least 90% of the first carrier should evaporate before the composition reaches the support surface but less than 50% of the second carrier should evaporate.

During spraying the spray should be fine so that the first carrier can evaporate from the composition and the fibres are able to conjugate during spraying.

Preferably, the container has a nozzle with internal diameter of 0.05 to 2 mm, more preferably 0.1 to 1 mm. This provides the fine spray. Preferably, conjugated fibres form during step b) so that the average length of fibres in the formed non-woven fabric is greater than the average length of fibres in the sprayed composition. Preferably, the spray formed in step b) is a fine spray with droplet size of less than 1 mm.

There are interacting factors which determine the type of fabric produced by spraying the composition of the present invention.

Different first carriers can have an effect on the resulting fabric as can changes in the fibre and binder contents as well as the fibres and binders used.

Changes in the distance from the point of spraying to the supporting surface can lead to differences in the resulting fabric. When the spray point is close to the supporting surface the fabric adheres more closely to the supporting surface. If the spray point is too close to the supporting surface insufficient propellant evaporates before the spray hits the surface a non-woven fabric is not produced but a film rather than a fabric is formed. Preferably, the distance between the spray point and the supporting surface is greater than 10 cm, more preferably greater than 30 cms. It has been found that if the amount of first carrier is increased the optimum spraying distance increases. Similarly, likewise, as the boiling point of the first carrier increases the optimum spraying distance increases.

When the spray point is further away from the supporting surface the non-woven fabric which forms is easily peeled from the support surface so that the fabric remains as a coherent layer. Preferably, the non-woven fabric is easily separated from the supporting surface.

The velocity of the composition when it leaves the spray point and when it arrives at the supporting surface can also affect the resulting fabric.

The methods and compositions of the invention may be used for a wide range of applications, some of which are listed below:

1. Direct spraying onto the human body e.g. dressings.
2. Bandages e.g. burns bandages or fracture casts
3. Spray-on fabric technology can be sprayed by spray-jet for industrial applications (e.g. J-cloths, shoe-shine cloths etc).
4. The technology can be used in conjunction with other additives of formulations, such as
Particles
Absorbing UV particles
Metal powder
Anti-bacterial agents
Sunscreens
Fragrances
Pigment and dyes
Lotions transferred to the skin to moisture and heal etc.
   5. Can also be used for the following technologies:
Polymer technology
Nano-technology
Bio-chips
Medical
Composites
Tissue engineering.

### EXAMPLE 1

### Instant Wet Spray Fabric Formulation

| Component | Ingredients | Quantities |
|---|---|---|
| Second carrier | Water | 29 g |
| First carrier | Diethyl ether | 72 g |
| Emulsifier/additive | Span 85 (surfactant) | 0.5 g |
| Emulsifier/additive | Tween 20 (surfactant) | 0.55 g |
| Binder | PS-PB-PS (Polystyrene- | 5 g |
| | polybutyrene-polystyrene block | |
| | copolymer) | |
| Fibres | Either Cotton CD60 fibres | 5.0 g |
| | Or Polypropene fibres | |
| | Or Polyamide fibres | |
| | Or mixtures thereof | |
| Additive | Silicone oil | 1 g |

The composition is prepared in two stages. Firstly, on emulsion of water in diethyl ether is formed by mixing the water, diethyl ether, Span 85 and Tween 20 and homogenising the mixture at 30,000 rpm for 5 minutes.

Secondly, the binder and fibres with silicone oil as a lubricant are added to the emulsion and the binder is dissolved in the diethyl ether phase of the emulsion by stirring for 12 hours.

1. A composition for forming a fabric by spraying onto a supporting surface, the composition comprising fibres, a binder, a first carrier in which the binder is dissolved and a second carrier wherein the boiling point of the second carrier is at least 40° C. and is higher than the boiling point of the first carrier.

2. A composition according to claim 1 wherein the boiling point of the second carrier is at least 30° C. higher than the boiling point of the first carrier, preferably at least 50° C. higher, more preferably at least 60° C. higher.

3. A composition according to claim 1 or 2 wherein the first carrier is selected from the group consisting of diethyl ether, dimethyl ether, liquefied petroleum gas, a C₁ to C₁₂ alcohol, a C₁ to C₁₂ ketone, a C₁ to C₁₂ ester, a C₅ to C₁₂ hydrocarbon or a mixture thereof.

4. A composition according to any preceding claim wherein the second carrier is selected from the group consisting of water, a ketone, an alcohol, a polyhydric alcohol, glycol ether or a mixture thereof.

5. A composition according to any preceding claim wherein the second carrier comprises water.

6. A composition according to any preceding claim wherein the first carrier and the second carrier are not miscible and the composition comprises an emulsion.

7. A composition according to claim 6 wherein the composition comprises an emulsion of second carrier in first carrier.

8. A composition according to any preceding claim wherein the first carrier comprises 10 to 80% by weight of the composition based on the total weight, preferably 20 to 50%.

9. A composition according to any preceding claim wherein the second carrier comprises 5 to 60% by weight of the composition based on total weight, preferably 10 to 50%.

10. A composition according to any preceding claim wherein the composition additionally comprises at least one additive selected from the group consisting of a cleaning agent, a drug, a moisturiser, a perfume or a dye.

11. A composition according to claim 10 wherein the additive is soluble in the second carrier.

12. A composition according to any preceding claim wherein the ratio of fibres to binder is in the range 1.5:1 to 1:1.5.

13. A composition according to any preceding claim wherein at least 80%, preferably at least 90%, more preferably at least 95% and most preferably substantially all of the fibres in the composition have a length of at least 0.02 mm.

14. A composition according to any preceding claim wherein at least 80%, preferably at least 90%, more preferably at least 95% and most preferably substantially all of the fibres have a length of not more than 10 mm.

15. An apparatus comprising a container containing the composition according to any preceding claim which is capable of producing a spray of the composition from the container.

16. An apparatus according to claim 15 wherein the composition in the container is at a pressure of above 1 and below 20 atmospheres, preferably between 5 and 20 atmospheres, more preferably between 8 and 15 atmospheres.

17. An apparatus according to claim 15 or 16, wherein the container has a nozzle with a diameter of 0.05 to 2 mm, preferably 0.1 to 1 mm.

18. A non-woven fabric produced by spraying a composition according to any of claims 1 to 11, wherein the fabric comprises the binder in solid form, fibres bound by the binder and second carrier in liquid form.

19. A non-woven fabric according to claim 18 wherein the fabric comprises at least 50% of the second carrier which was present in the composition before spraying and less than 10% of the first carrier which was present in the composition before spraying.

20. A non-woven fabric according to claim 18 or 19 wherein the fabric additionally comprises an additive.

21. An article comprising a non-woven fabric according to any one of claims 18 to 20.

22. An article according to claim 21, wherein the article is a medical dressing, a cleaning cloth or a cosmetic applicator.

23. A method for producing a non-woven fabric comprising
a) providing a container capable of producing a spray containing a composition according to any of claims 1 to 10, and
b) spraying the composition onto a supporting surface thereby forming a non-woven fabric on the supporting surface.

24. A method according to claim 23 wherein in step b), at least 90% of the first carrier and less than 50% of the second carrier evaporates before the composition reaches the supporting surface.
